(19) United States
(12) Patent Application Publication
(10) Pub. No.: US 2005/0033157 A1
(43) Pub. Date: Feb. 10, 2005

(54) Multi-modality marking material and method

(76) Inventors: Dean A. Klein, North Oaks, MN (US); James D. Brazil, Braham, MN (US); Thomas M. Jaeger, Minnetonka, MN (US); Daniel A. Halpern, St. Louis Park, MN (US); Mark W. Gillick, Edina, MN (US)

Correspondence Address: John L. Crimmins;FAEGRE & BENSON LLP, 2200 Wells Fargo Center, 90 South Seventh Street, Minneapolis, MN, 55402-3901, US

(21) Appl. No.: 10/626,931

(22) Filed: Jul. 25, 2003

(51) Int. Cl. A61B 5/05

(52) U.S. Cl. 600/411

(57) ABSTRACT

The present invention provides markers and methods of using markers to identify or treat anatomical sites in a variety of medical processes, procedures and treatments. The markers of embodiments of the present invention are permanently implantable, and are detectable in and compatible with images formed by at least two imaging modalities, wherein one of the imaging modalities is a magnetic field imaging modality. Images of anatomical sites marked according to embodiments of the present invention may be formed using various imaging modalities to provide information about the anatomical sites.

### BACKGROUND

Minimally invasive medical treatment techniques are becoming an increasingly prominent method of performing procedures for the diagnosis, treatment and/or monitoring of conditions, which were traditionally performed through an open incision. The adoption of these techniques has been made possible by the development of imaging techniques and systems that allow clinicians to obtain views or images of the anatomical features of portions of the human body. Imaging techniques and systems including computed tomographic X-ray (CT) imaging, portal film imaging devices, electronic portal imaging devices, electrical impedance tomography (EIT), nuclear medicine (NM) such as positron emission tomography (PET) and single photon emission computed tomography (SPECT), magnetic source imaging (MSI), magnetic resonance spectroscopy (MRS), laser optical imaging, magnetic resonance imaging (MRI), magnetic resonance mammography (MR mammography), electric potential tomography (EPT), brain electrical activity mapping (BEAM), magnetic resonance angiography (MRA), magnetoelectro-encephalography (MEG), arterial contrast injection angiography and digital subtraction angiography, have provided clinicians with improved visualization of the anatomical structure of portions of the human body without having to perform invasive surgical techniques. These advanced techniques are also being integrated with more traditional imaging modalities, such as X-ray (e.g. mammography, fluoroscopy and kV X-ray), ultrasound and video imaging.

The use of one or more of the above-described imaging modalities for obtaining and analyzing anatomical structures is becoming increasingly prominent in many medical procedures. For example, in the field of neurosurgery, prior to performing surgery, a three-dimensional image of a patient's head may be formed using an imaging modality such as a CT imaging system. The CT image may be used by the surgeon in planning the surgery and for establishing a three-dimensional frame of reference for the operation. In another example, these imaging modalities may be used in the field of oncology for the identification, planning, staging, treatment and monitoring of lesions or other areas of abnormal tissue. For example, imaging modalities currently used in the diagnosis and monitoring of breast lesions for example, include mammography, ultrasound, and increasingly, MRI and/or MR mammography. These imaging modalities may be critical in treating lesions by, for example, chemotherapy, surgery and radiation therapy. For example, when a patient is treated with chemotherapy, drugs are introduced into the patient's body to destroy the lesion. During the course of this treatment, a variety of imaging modalities may be implemented to follow the progress of the treatment or condition by comparing a series of images of a particular treatment site over time. In another example, positional information obtained from the images may be used before or during the performance of a medical procedure at the site of the lesion. In yet another example, after a lesion is removed by surgical methods, one or more imaging modalities may be useful in imaging the site of lesion removal to monitor the condition of the site.

The use of these imaging modalities may be particularly important in the success of radiation therapy. Radiation therapy involves subjecting a lesion to X-ray or electron radiation through the use of, for example, a linear accelerator. In radiation therapy, geometric accuracy is a very important factor to the success of treatments. The goal of radiation therapy is to hit a specific target (i.e. a lesion), without hitting healthy tissue. A critical factor to precisely targeting a lesion site and avoiding healthy tissue is proper positioning of a patient in reference to the radiation-producing apparatus. The use of one or more imaging modalities has become an important component in properly positioning a patient for radiation therapy because such imaging may provide multiple data sets for positioning the patient and may provide for improved patient positioning over multiple treatments.

An increasingly important factor in utilizing these various imaging modalities for non-invasive medical procedures is the ability to interpret, compare, synthesize, fuse, and/or to integrate images to obtain positional information about a portion of the body or an anatomical site of a patient. Such “body mapping” or “multi-modality image fusion” techniques use various data points or positional locators on or in the body in order to pinpoint the exact location in which a particular technique is to be performed. For example, body positioning techniques for radiation therapy often involve taking a reference image of a patient's body positioning prior to radiation therapy, and then visually comparing or electronically integrating or synthesizing the reference image with subsequent images of a patient's body position in order to properly position the patient each time radiation therapy is performed.

Problems associated with the imaging techniques mentioned above include both the accurate selection and the comparison of views of identical areas in images that have been obtained at different times or by images obtained using different image modalities. These problems have at least two aspects. First, in order to relate information in an image of the anatomy to the anatomy itself, it is beneficial to establish one-to-one mapping between points in the image and points on the anatomy. This is referred to as registering image space to physical space.

The second aspect concerns the registration of one image space to another image space. The goal of registering two arbitrarily oriented three dimensional images is to align the coordinate systems of the two images such that any given point in the scanned anatomy is assigned identical addresses in both images. The calculation of the rigid body transformation necessary to register the two coordinate systems requires knowledge of the coordinate vectors of at least three points in the two systems. Such points are called “fiducial points” or “fiducials,” and the fiducials used are the geometric centers of markers, which are called “fiducial markers”. These fiducial markers are used to correlate image space to physical space and to correlate one image space to another image space. The fiducial markers also provide a constant frame of reference visible in a given imaging modality to make registration possible.

A variety of techniques have been developed to improve body mapping and body positioning such that the accuracy of treatments is increased. U.S. Pat. No. 6,314,310 to Ben-Haim et al. reports an apparatus for X-ray guided surgery including a reference element having a plurality of fiducial marks, a first coordinate sensing device and a surgical tool having a second coordinate sensing device. A fluoroscope forms an X-ray image of the body, including the fiducial marks. A computer analyzes the image to determine the position of the reference element in the image so as to find coordinates of the first coordinate sensing device relative to the image, and registers the position of the tool with the X-ray image by referring to coordinates of the second coordinate sensing device to the known coordinates of the first position sensor.

U.S. Pat. No. 6,405,072 to Cosman reports a system for positioning and repositioning a portion of a patient's body including multiple cameras to view the body and index markers that may be located by the cameras. X-ray imaging of the patient further refines the anatomical target relative to a treatment or diagnostic imaging reference point. U.S. Pat. No. 6,359,960 to Wahl et al. reports a method for automatically determining coordinates relative to a reference coordinate system of radiopaque markers.

U.S. Pat. No. 6,516,046 to Frohlich et al. reports a method for exact positioning of a patient for radiotherapy or radiosurgery, in which a patient is pre-positioned relative to a linear accelerator, and then an X-ray image of the patient is taken in the vicinity of the radiation treatment target. The resulting image is mapped, and then a reconstructed image is generated from a three-dimensional set of patient scanning data corresponding to the X-ray image. The reconstructed image is then superimposed on the X-ray image to detect positional errors based on specific landmarks (e.g. natural landmarks and skin markers) on both images. The position of the patient is then corrected on the basis of the positional errors.

U.S. Pat. No. 6,351,573 to Schneider reports a method and apparatus for obtaining and displaying in real time an image of an object obtained by one modality such that the image corresponds to a line of view established by another modality.

These references report the utilization of reference points on or within the body in order to create data or mapping points as part of the body mapping, positioning and/or treatment process. These reference points are generally either anatomical parts or structures, or markers (e.g. fiducial markers or tissue markers) positioned on or inside a patient.

The use of markers placed on or inside a patient may be particularly useful because the markers may provide a constant frame of reference visible in one or more imaging modes. In this manner, markers may reduce error caused by movement of body parts otherwise used as reference points.

A marker commonly used in biopsy procedures includes a metallic clip (e.g., a clip sold under the trade name Micromark™, from Johnson & Johnson) delivered through a 9-, 11- or 14-gauge probe of a biopsy device, and attached to the site of a biopsy to mark the location of the biopsy. These clips are approximately 3 mm across and are permanent and radiopaque. The use of marking clips has also been reported in Burbank et. al., “Tissue Marking Clip for Stereotactic Breast Biopsy: Initial Placement Accuracy, Long-term Stability, and Usefulness as a Guide for Wire Localization,” Radiology 1997; 205:407-415; and Liberman et. al., “Clip Placement After Stereotactic Vacuum-Assisted Breast Biopsy,” Radiology, 1997; 205:417-422.

U.S. Pat. No. 6,394,965 to Klein reports a method of tissue marking using microparticles having a carbon surface. In one embodiment, the microparticles include a radiopaque material and a pyrolytic carbon surface.

Other markers are reported in U.S. Pat. Nos. 6,333,971, 4,222,499, 5,397,329, 6,351,573, 6,419,680, 6,516,046, and 6,381,485, as well as U.S. Published Patent Application Nos. 2002/018896, 2002/0143357, 2002/0035324, 2002/017437, and 2003/0086535.

One difficulty in the use of markers for procedures utilizing multiple imaging modalities is that a marker that is detectable in and compatible with one imaging modality (e.g. X-ray) may not be detectable in or compatible with another imaging modality (e.g. MRI). For example, the marker may not be detectable in images formed by the other imaging modality. Alternatively, the marker may be detectable, but may cause substantial distortion or interference with images formed by certain imaging modalities. Furthermore, certain markers may pose a safety risk to a patient exposed to certain imaging modalities such as MRI imaging modalities.

For example, conventional markers such as stainless steel or titanium markers, may be detectable in and compatible with X-ray and other non-magnetic field imaging modalities, but may not be compatible with images produced via magnetic field imaging modalities such as MRI. More specifically, the interaction of the magnetic and/or conductive properties of the marker with the magnetic field applied during MRI causes image distortion. Image distortion may be caused by three general classes of interactions with the applied magnetic field: static magnetic field distortions (e.g., inhomogeneities caused by induced magnetization), dynamic distortions (e.g., magnetic fields caused by gradient induced eddy-currents) and RF field non-uniformities (e.g., secondary fields induced by conducting structures). These classes of image distortion are collectively referred to herein as “image distortion.” Image distortion may be particularly notable with markers containing ferromagnetic materials, paramagnetic materials or other materials of high magnetic susceptibility (i.e., the response of a material to an applied magnetic field). These materials also may pose a safety risk associated with the exposure of the marker to external or applied magnetic fields, such as movement of the marker within the body.

Thus, it would be advantageous to provide a biocompatible marker, particularly a permanent biocompatible marker, which is detectable in and compatible with both magnetic and non-magnetic field imaging modalities such that images from one or more imaging modalities may be obtained for use in a variety of medical procedures.

### SUMMARY OF THE INVENTION

In one embodiment, the present invention provides a method for identifying an anatomical site to be treated, in which at least one permanent marker is implanted at the anatomical site. The marker includes a solid material that is both detectable in and compatible with images formed by at least two imaging modalities, wherein one of the modalities is a magnetic field imaging modality. At least one image of the anatomical site, in which the marker is detectable and compatible, is formed to obtain information about the anatomical site. The anatomical site may then be treated using the information obtained from the image(s).

As used herein, the phrase “magnetic field imaging modality” refers to imaging modalities formed by measuring magnetic fields in the body, or by measuring the reaction of the body to the application of a magnetic field. Representative examples of magnetic field imaging modalities include MRI, MSI, MRS, MEG, MSA and MRA.

The markers of the present invention are also detectable in and compatible with one or more non-magnetic field imaging modalities, including radiation imaging modalities and ultrasound imaging modalities. An example of a radiation imaging modality is X-ray imaging, including computed tomography, fluoroscopy and mammography. Other non-magnetic field imaging modalities that may be suitable for use in embodiments of the present invention include NM (e.g. PET or SPECT), EIT, EPT, BEAM, EPID, laser optical imaging, arterial contrast injection angiography, digital subtraction angiography, video imaging and ultrasound imaging. In a particular embodiment, the marker is detectable in and compatible with images formed by at least 3 imaging modalities, including magnetic field imaging, radiation (e.g. X-ray) and ultrasound imaging modalities.

As used herein, the term “detectable” refers to a marker that can be recognized or visualized in images formed by a particular imaging modality. Such detection may include visualization (e.g. recognition by the human eye), recognition and/or interpretation by a computer system or other automated system, or a combination thereof. As used herein, the term “compatible” refers to a marker that does not cause substantial image distortion, image artifacts, spectral distortion, spectral artifacts or otherwise compromise or adversely affect the use and/or interpretation of the image to obtain information about the anatomical site (or other sites) being imaged.

Information that may be obtained from the image or images includes diagnostic information, positional information, condition information and/or other information that may be useful to a clinician in treating a patient. For example, information obtained from images may assist a clinician in diagnosing a condition at a tissue site. Images may also provide a clinician with information relating to the relative position of an anatomical site within the body before or while performing a medical procedure. Additionally, images taken may provide information about the condition of the treatment site, such as the success of treatment, or the progression of a condition.

Information obtained from the image(s) may be used in a variety of treatments. In one embodiment, treating the anatomical site includes monitoring the anatomical site using the information obtained from the image(s). In another embodiment, treating the anatomical site may include a medical procedure, such as radiation therapy, drug therapy or surgery (e.g. lesion removal).

The marker of embodiments of the present invention may be implanted at a variety of anatomical sites, including tissue removal sites, biopsy sites (e.g. breast or prostate), polyp sites, lesion sites or other sites of interest. The marker may be permanently implantable such that the marker will remain permanently at the tissue site unless intentionally removed.

Embodiments of the present invention may also utilize a carrier with the marker. For example, before, after or while implanting one or more markers into a biopsy site, a carrier may be injected at the anatomical site. The carrier may be a biologically compatible solution, such as a suspension, dispersion or other fluid or gel. In one embodiment, the carrier is a solution including β-glucan or a derivative thereof.

In another embodiment, the present invention provides a method of multi-modality fusion for mapping a portion of a body, in which a permanent marker is implanted into the body. The marker includes a solid material that is detectable in and compatible with images formed by at least two imaging modalities, wherein one of the modalities is a magnetic field imaging modality. First and second images, in which the marker is detectable and compatible, are then formed using first and second imaging modalities, and at least one of the first and second imaging modalities is a magnetic field imaging modality. The first and second images are then synthesized, for example, by a suitable computer system, to obtain information for a portion of the body.

A further embodiment of the present provides a method of positioning a body for radiation therapy. After selecting an anatomical site upon which radiation therapy is to be performed, a permanent marker is implanted at the anatomical site. The marker includes a solid material that is detectable in and compatible with images formed by at least two imaging modalities, wherein one of the modalities is a magnetic field imaging modality. An image, in which the marker is detectable and compatible, is then formed to obtain information about the anatomical site. The body may then be positioned for radiation therapy based on information provided by the first image. Optionally, at least a second image of the treatment site may then be formed before, during or after positioning the body. Information obtained from the first and second images may be compared prior to performing radiation therapy on the treatment site, and the body may then be re-positioned as needed. Additional images may be formed during subsequent radiation therapy sessions.

Further yet, an embodiment of the present invention provides a method of identifying a lesion site of the breast for treatment, in which at least one marker is implanted. The marker may be formed from a solid material that is detectable in and compatible with images formed from at least two imaging modalities. An image of the lesion site, in which the marker is detectable and identifiable, may then be formed to obtain information about the lesion site. The lesion site may then be treated by monitoring, or via a medical procedure based on information provided by the image of the lesion site.

In yet a further embodiment, the present invention provides a method for computer assisted diagnosis to provide diagnostic information about a patient, in which a marker is implanted into a patient. The marker is formed of a material that is not categorized as abnormal tissue by computer assisted diagnosis systems. Computer assisted diagnosis is then performed on the patient.

The present invention also provides a permanently implantable biocompatible marker including at least one solid material that is detectable in and compatible with images formed by at least two imaging modalities, one of the imaging modalities being a magnetic field imaging modality. The marker may be shaped to be distinguishable from anatomical structures in images formed by the magnetic field imaging modality.

Suitable marker materials for embodiments of the present invention include graphite, and ceramic materials such as zirconium oxide, aluminum oxide, hydroxyapatite and silicon dioxide. The marker material may also be coated with a biocompatible coating, such as carbon or a carbon resin. Carbon coated zirconium oxide may be particularly useful for embodiments of the present invention.

The marker may be sized and shaped in a variety of ways to be distinguishable from anatomical structures. In one embodiment, the marker has a major dimension between about 80 and about 10,000 microns more particularly between about 800 and about 3,500 microns. In another embodiment, the marker is formed in a “barbell” or “dog bone” configuration.

Optionally, the marker may include additional materials to enhance the multi-modality imaging characteristics of the marker. In one embodiment, the marker may incorporate material sensitive to an additional imaging modality such as electronic portal imaging or portal film imaging. For example, the marker may include radiopaque material such as gold, titanium, platinum, palladium, gadolinium, tantalum or a polymer. The marker may also include a biologically active agent, for example a biologically active gel, if desired.

In yet a further embodiment, the present invention provides a kit including the at least one marker formed according to the embodiments reported herein and a carrier solution for delivery to a desired site. Suitable carriers include solutions of β-glucan and collagen.

### BRIEF DESCRIPTION OF THE DRAWINGS

FIG. 1 illustrates a magnetic resonance image of Marker A, a stainless steel marker available from Johnson and Johnson under the trade name Mammotome.

FIG. 2 illustrates a magnetic resonance image of Marker B, a stainless steel marker available from SenoRx, Aliso Viejo, Calif. under the trade name Ultracor.

FIG. 3 illustrates a magnetic resonance image of Marker C, a stainless steel marker available from SenoRx, Aliso Viejo, Calif. under the trade name Biopsy site Marker.

FIG. 4 illustrates a magnetic resonance image of Marker D, a titanium alloy marker available from Artemis, Hayward, Calif.

FIG. 5 illustrates a magnetic resonance image of Marker E, a stainless steel alloy marker available from Inrad, Kentwood, Mich.

FIG. 6 illustrates a magnetic resonance image of Marker F, a carbon coated zirconium oxide marker included in one embodiment of the present invention.

### DETAILED DESCRIPTION

The present invention provides markers for use in medical procedures that may benefit from the use of multi-modality imaging procedures. The markers of the present invention are detectable in and compatible with both magnetic field imaging modalities and non-magnetic field imaging modalities. Representative examples of magnetic field imaging modalities include MRI, MSI, MRS, MEG, MRA, MSA and MR mammography. MRI may be particularly suitable for use with embodiments of the present invention. Suitable MRI systems include T3 and T4 scanners commonly used for clinical patient examinations. Such scanners are manufactured by, for example, Siemens AG and may include a 90 cm diameter whole-body superconducting magnet equipped with head RF coils. Another example of a suitable system is a Phillips 4T MRI/MRS scanner.

Representative examples of non-magnetic field imaging modalities include radiation and ultrasound imaging modalities. Particular examples of radiation imaging modalities include X-ray imaging such as CT, fluoroscopy and mammography. Other non-magnetic field imaging modalities may include NM (e.g. PET and SPECT), EIT, EPT, BEAM, electronic portal imaging, portal film imaging, laser optical imaging, arterial contrast injection angiography, digital subtraction angiography, and video imaging.

In one embodiment, the markers may be detectable in and compatible with images formed by magnetic field, X-ray and ultrasound imaging modalities. Images formed by the various modalities may provide information about an anatomical site, such as diagnostic information, positional information, or condition information (e.g. success of treatment or progression of condition). This information may then be used to make treatment decisions, and/or to perform medical procedures.

Markers formed according to embodiments of the present invention include graphite, and ceramic materials such as zirconium oxide, aluminum oxide, silicon dioxide, and hydroxyapatite. In a further embodiment, the marker may be composed of a substrate including the aforementioned materials and a biocompatible coating such as a pyrolytic carbon, vitreous carbon, graphite or a carbon resin coating. In one embodiment, the marker may include a ceramic zirconium oxide substrate coated with pyrolytic carbon.

Pyrolytic carbon coatings may be produced and coated onto substrate surfaces by known methods. In one technique, hydrocarbons and alloying gases are decomposed to prepare a pyrolytic carbon coating on the substrate. The substrate is contacted with the hydrocarbons and alloying gases in a fluidized or floating bed at a temperature sufficient to cause deposition of pyrolyzed carbon onto the substrate surface, typically from about 900 to 1500° C. Inert gas flow is used to float the bed of substrates, optionally including an inert mixing media. The hydrocarbon pyrolysis results in a high carbon, low hydrogen content carbon material being deposited as a solid layer of material onto the substrate.

Alternatively, in another embodiment, a carbon coating (sometimes referred to as “ultra-low-temperature isotropic carbon”) may be applied to substrate using any one of other various coating processes for depositing carbon, such as a vacuum vapor deposition process. Such a method uses ion beams generated from any of a variety of known processes, such as the disassociation of CO₂, reactive dissociation in vacuum of a hydrocarbon as a result of a glow discharge, sublimation of a solid graphite source, or cathode sputtering of a graphite source. Ceramics, zirconium, graphite or titanium substrates may be suitable for this type of coating process.

Isotropic carbon may also be applied to temperature-sensitive substrates using physical vapor deposition techniques. Physical vapor deposition involves transferring groups of carbon atoms from a pyrolytic carbon target to a desired substrate at low temperatures. The process may be carried out in high-vacuum conditions to prevent chemical reaction. This technique may be suitable for coating a variety of temperature-sensitive substrates, such as certain polymeric materials.

The high strength, resistance to breakdown or corrosion, and durability of a carbon surface ensures effective, long term functioning of coated substrates in marking applications. The established biocompatibility of carbon coatings such as pyrolytic and vitreous carbon coatings makes the described markers particularly suitable for marking applications. The substrate may be completely encased by a carbon surface. This results in a uniformly coated marker with no substrate exposure on the surface of the particle. Preferred carbon coatings may be in the range of fractions of thousandths of an inch, e.g., about ½ of a thousands of an inch (0.0005 inch), on average, covering the surface of the substrate.

The marker of the present invention may be sized as desired for a particular marking application. In one embodiment, for example, the marker may have a major dimension (e.g., diameter or length) of at least about 80 microns, more particularly between about 800 and about 10,000 microns, even more particularly between about 800 and about 3500 microns, and even more particularly between about 1000 and about 3000 microns.

A wide variety of marker shapes may be suitable for use in the present invention. Particularly suitable marker shapes may be readily distinguishable from anatomical features of a patient and lines of calcification in images formed by both magnetic and non-magnetic field imaging modalities. In one embodiment, the marker may be formed in a “barbell” or “dog bone” configuration. Other suitable shapes may include hollow or solid rods, spheres, coils, helixes, circular or oval rings, hollow or solid tubes and various combinations thereof.

Additional components may be added to embodiments of the present invention such that the markers may be detectable in additional imaging modalities. For example, certain embodiments may incorporate a layer of gold, titanium, platinum, palladium, gadolinium, tantalum or a polymer material to provide for enhanced compatibility with electronic portal imaging, portal film imaging or other imaging modalities. Alternative additional components include liquids that may be disposed in hollow portions of embodiments of the present invention.

The markers of the present invention may also incorporate a bioactive agent, including an anti-inflammatory, an anti-microbial, a hemostatic agent, a biocompatible adhesive agent, a protein, a stem cell or other cell-derived material. In one embodiment, the bioactive agent is formed as a bioactive gel, which may be applied onto a surface of the marker. In another embodiment, the bioactive agent may be disposed within a hollow portion or cavity in or on the marker.

The markers of the present invention may be implanted in a variety of conventional manners. In one embodiment, the marker may be implanted as part of a non-invasive medical procedure. For example, the marker may be implanted during a non-invasive tissue removal procedure or a biopsy procedure. In another embodiment, a biopsy system may be fitted with a device for implanting the marker. In a further embodiment, the marker may be implanted using a suitable needle. Alternatively, the marker may be implanted via conventional surgical methods.

Furthermore, during implantation, the marker of the present invention may be guided to a desired anatomical site by utilizing one or more imaging modalities in which the marker is detectable. Suitable modalities for guiding implantation of the marker include magnetic resonance, radiation and ultrasound imaging modalities.

The markers of the present invention may be suitable for use in a variety of procedures or treatments that involve imaging a particular anatomical site. The markers may be particularly useful in the field of oncology for treating lesions or other abnormal tissue sites. As used herein, the term “treating” refers to a broad range of activities in which identifying an anatomical site is desirable, including monitoring an anatomical site, staging and planning for medical procedures, performing medical procedures (e.g., radiation therapy, biopsy, surgery, drug therapy, RF ablation, and radiotherapy), and evaluating the success of a particular treatment.

For example, a lesion or other abnormality at or in an anatomical site is often discovered during a routine exam, or from an image formed of the anatomical site. After discovering a lesion, it may be desirable for a clinician to mark the anatomical site by implanting a marker. This implantation step may occur as a separate procedure or during a biopsy or other tissue removal procedure in order to perform tests on the lesion.

After implantation of the marker of the present invention, one or more imaging modalities, in which the marker is detectable and compatible, may be used to form one or more images of the anatomical site. The images may be used to obtain further information about the anatomical site, and the information may then be used to treat the anatomical site.

In one example, the clinician may determine that the lesion is benign, or does not otherwise pose an immediate health risk. However, the clinician may wish to monitor the anatomical site for any progression or change in the lesion over time. Advantageously, the marker of the present invention is not only permanent, but is detectable in and compatible with images formed from a variety of imaging modalities such that the clinician can obtain images from multiple modalities if desired. Additionally, in the event that an image of the anatomical site is desired for reasons unrelated to the lesion, the marker is detectable in and compatible with MRI, X-ray, ultrasound and other imaging modalities.

In another example, the clinician may determine that the lesion or abnormality should be treated, for example, by surgical removal, drug therapy or radiation therapy. In this example, information obtained from images may be used to determine the exact position of the lesion for treatment, and/or to monitor the success of a particular treatment.

In yet another embodiment, the clinician may discover and remove a lesion without first performing a biopsy. In this case, one or more markers formed according to embodiments of the present invention may be implanted at the lesion site prior to removal to guide the procedure, or after removal for future monitoring via one or more imaging modalities.

Embodiments of the present invention including zirconium oxide markers may be particularly useful for marking the site of breast biopsies. The most common imaging modalities used to form images of breast biopsy sites are currently MRI, mammography (MR and X-ray) and ultrasound. Advantageously, embodiments of the present invention are detectable in and compatible with all of these imaging modalities.

In yet another example, one or more markers may be implanted at an anatomical site to enhance multi-modality fusion, for example, in oncology planning, staging, treatment and monitoring procedures. After implantation of one or more markers, positional information about the anatomical site may be obtained by the synthesis of a plurality of imaging modalities in which the markers are detectable and compatible. As used herein, the term “synthesizing” refers to the integration or fusion of two or more images, formed by different imaging modalities, into a set of data points. An example of a suitable system for synthesizing multiple images of a body is reported in U.S. Pat. No. 6,351,573 to Schneider, incorporated herein by reference. Schneider reports an apparatus for obtaining and displaying in real time an image of an object obtained by one modality such that the image corresponds to a line of view established by another modality. The markers of the present invention may be particularly useful for incorporation into such systems because the markers are detectable in and compatible with both magnetic and non-magnetic field imaging modalities, such as X-ray and ultrasound imaging modalities. Representative examples of imaging modalities that may be successfully fused include MRI, CT X-ray, PET and NM. Particular combinations for fusion include CT/MRI, NM/MRI/CT and PET/CT.

In a further example, one or more markers may be suitable for use in radiation therapy procedures. For example, after selecting an anatomical site to be treated by radiation therapy, one or more markers formed according to embodiments of the present invention may be implanted at the anatomical site. At least one image of the anatomical site may then be formed using an imaging modality in which the marker is detectable and compatible. The resulting image(s) may then be used as a basis for positioning the patient for a radiation therapy session.

The implanted markers may be particularly useful if a clinician desires to position a patient for radiation therapy using multiple imaging modalities. For example, a first image of a marked anatomical site may be formed using MRI to provide comprehensive positional information. The patient could then be positioned for radiation therapy. A second image of the marked anatomical site could then be formed using a more conventional imaging method, for example, X-ray or ultrasound, while the patient is positioned for radiation therapy. Positional information provided by the images could then be compared, utilizing the fact that the markers are compatible with both imaging techniques. Any positional difference between the two images could then be corrected, reducing the degree of error in the radiation therapy procedure. This method may also be useful for positioning a patient over multiple radiation therapy sessions.

U.S. Pat. No. 6,516,046 to Frohlich et al, incorporated herein by reference, reports a method for positioning a patient for radiotherapy, in which a patient is positioned relative to a linear accelerator (e.g. portal film imaging or electronic portal imaging) to produce an X-ray image of the patient that is subsequently mapped. A reconstructed image is then generated from a three-dimensional set of patient scanning date formed, for example, as digitally reconstructed radiographs. The two images are then superimposed, and positional differences between the images are detected to allow for correction of the patient's position. The markers of the present invention may also be suitable for incorporation into the method reported in Frohlich and similar methods.

In certain embodiments, the markers of the present invention may be compatible with Computer Assisted Diagnosis (CAD) systems. CAD systems analyze images from a variety of image modalities and then identify and/or classify abnormal tissue. Such classifications assist doctors in analyzing images and making a diagnosis. Further details about CAD systems are reported in U.S. Pat. No. 6,301,378 to Karssemeijer et al.

Unfortunately, the presence of conventional markers in images used in CAD systems may result in the markers being classified as being abnormal tissue, in essence resulting in a false positive diagnosis. However, the markers of embodiments of the present invention are not classified as abnormal tissue by CAD systems. In the near future, such CAD systems may be used to identify the marked abnormal tissue and to communicate with a radiation therapy system to treat the abnormal tissue.

In certain embodiments, a biocompatible carrier solution may be injected into a desired anatomical site before, subsequent to, or during the implantation of the marker. Suitable carriers include biologically compatible solutions, including solutions containing glucan, collagen, saline, dextrans, glycerol, polyethylene glycol, corn oil or safflower, other polysaccharides or biocompatible polymers, methyl cellulose, agarose, natural or synthetic proteins or combinations thereof. The carrier may also include a suitable hemostatic agent. The viscosity of the carrier ranges between about 10 and 75,000 centipoise.

Solutions containing β-glucan and collagen are particularly suitable carriers for embodiments of the present invention. β-glucan is a naturally occurring constituent of cell walls in essentially all living systems including plants, yeast, bacteria, and mammalian systems. Its effects and modulating actions on living systems have been reported by Abel et. al., “Stimulation of Human Monocyte B-glucan Receptors by Glucan Particles Induces Production of TNF-∂ and 1L-B,” Int. J. Immunopharmacol., 14(8):1363-1373, 1992. β-glucan, when administered in experimental studies, elicits and augments host defense mechanisms including the steps required to promote healing, thereby stimulating the reparative processes in the host system. β-glucan is removed from tissue sites through macrophagic phagocytosis or by enzymatic degradation by serous enzymes. The degradation or removal of β-glucan, as well as its available viscosity and lubricous nature, make it a useful carrier in marking applications.

Aqueous solutions of β-glucan may be produced that have favorable physical characteristics as a carrier solution in marking applications. The viscosity may vary from a thin liquid to a firm, self-supporting gel. Useful β-glucan compositions include β-D-glucans containing 4-0-linked-β-D-glycopyranosyl units and 3-0-linked-β-D-glycopyranosyl units, or 5-0-linked-β-D-glycopyranosyl units and 3-0-linked-β-D-glycopyranosyl units.

Collagen, another suitable carrier, is a naturally occurring protein that provides support to various parts of the human body, including the skin, joints, bone and ligaments. One suitable injectable collagen manufactured by the McGhan Medical Corporation, Santa Barbara, Calif., and sold under the trade names ZYDERM and ZYPLAST, is derived from purified bovine collagen. The purification process results in a product similar to human collagen. Collagen solutions may be produced within a wide viscosity range to meet an individual patient's needs, and have been shown to have a hemostatic effect.

Another example of a suitable carrier material is a solution containing methyl cellulose or another linear unbranched polysaccharide. Further examples of appropriate carrier materials include agarose, hyaluronic acid, polyvinyl pyrrolidone or a hydrogel derivative thereof, dextran or a hydrogel derivative thereof, glycerol, polyethylene glycol, oil-based emulsions such as corn or safflower, or other polysaccharides or biocompatible organic polymers either singly or in combination with one or more of the above-referenced solutions.

In certain embodiments, it may be desirable to include a hemostatic agent in the carrier. Suitable hemostatic agents may include substances derived from the blood such as collagen, fibrinogen, thrombin and other natural proteins, as well as a variety of synthetic proteins or other synthetic hemostatic agents.

### EXAMPLE

Markers A-F, each having a major dimension of 3 mm were placed 7 cm apart in a layered gelatin phantom (Knox brand flavorless gelatin, commercially available from Kraft Foods) for analysis. Markers A-C and E were composed of stainless steel alloys, marker D was composed of a titanium alloy, and Marker F was composed of a zirconium oxide substrate formed in a “dog bone” shape and coated with pyrolytic carbon.

The markers were then analyzed under ultrasound, mammography and MRI imaging modalities. The ultrasound was performed using a GE ultrasound system, mammography was performed using a Siemens system, and the MRI was performed on a Phillips 4T MRI/MRS scanner. The spatial extent of the MRI artifact was measured using a 3D FLASH image (TE/TR—6/17 ms, 0.4×1.7 mm resolution). Spectral distortion was measured by comparing linewidth of the water resonance from a 1 ml voxel centered on each marker, to the water linewidth measured in a control voxel containing no marker.

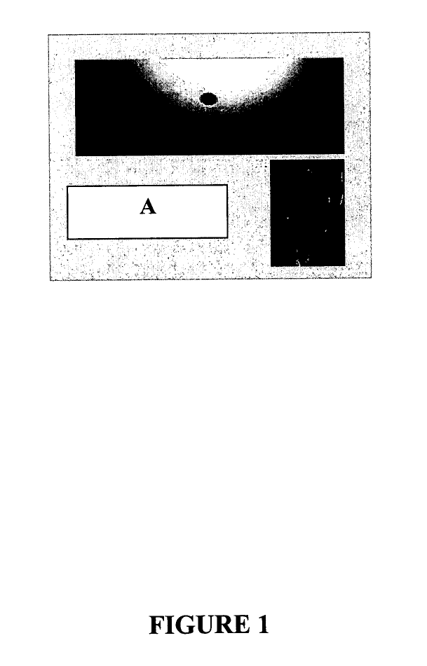

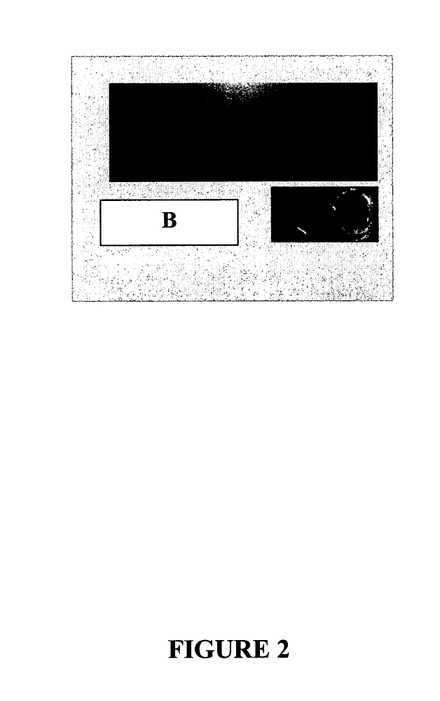

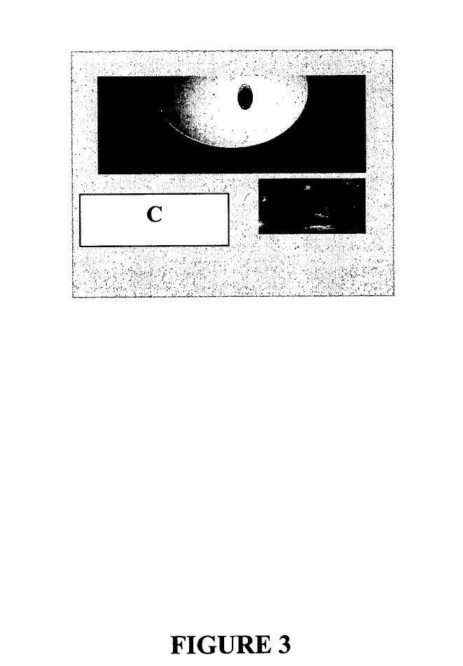

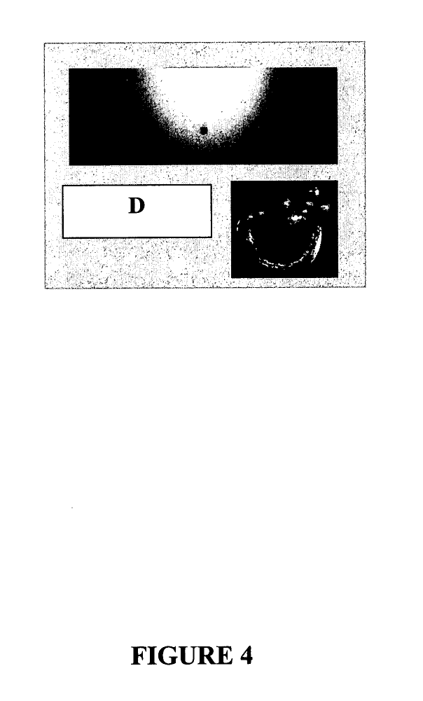

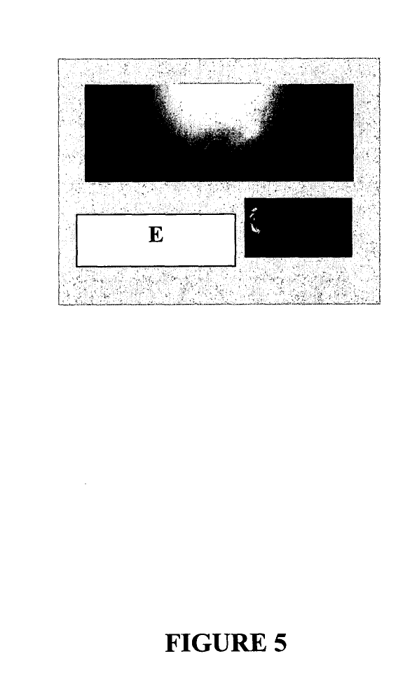

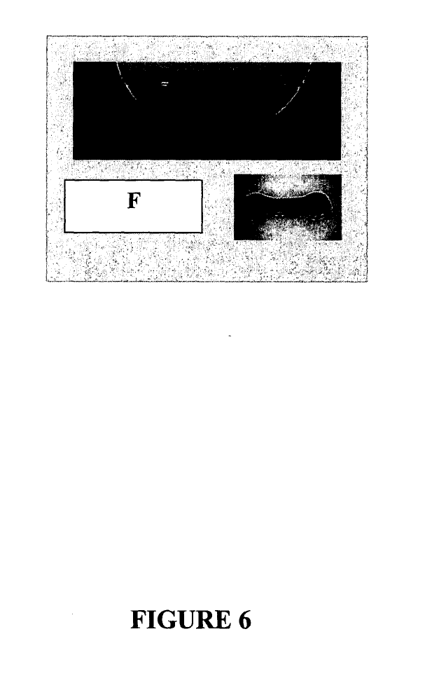

All six markers were detectable in and compatible with both ultrasound and mammography. However, as demonstrated in FIGS. 1-6 and Table 1 below, Markers A-E produced significant imaging artifacts and spectral artifacts compared to Marker F, which was formed according to an embodiment of the present invention.

TABLE 1

| Marker | A | B | C | D | E | F |
|---|---|---|---|---|---|---|
| Imaging | 14 mm | 17 mm | 17 mm | 10 mm | 27 mm | 3 mm |
| Artifact | | | | | | |
| Spectroscopic | 25.5 Hz | 14.2 Hz | 13.9 Hz | 44 Hz | 106 Hz | 9.4 Hz |
| Artifact | | | | | | |

Table 1 demonstrates that Markers A-C and E produced 14-28 mm of imaging artifact and Marker D produced 10 mm of imaging artifact. Marker F produced only a 3 mm imaging artifact, which is substantially equal to the size of the marker. Furthermore, spectral artifacts produced by Markers A-C and E ranged from 14-106 Hz and Marker D produced a spectral artifact of 44 Hz. In contrast, Marker F produced a spectral artifact of only 9.4 Hz.

This Example demonstrates that Marker F, the carbon coated zirconium oxide marker, is not only detectable in and compatible with ultrasound and mammography, but is also detectable in and compatible with MRI. Marker F also produced a low spectral artifact under MRS. In contrast, Markers A-E were significantly less compatible with MRI than Marker F and produced significantly higher spectral artifacts under MRS.

1. A method of identifying an anatomical site for treatment comprising:
implanting at least one permanent marker at the anatomical site, the marker comprising a solid material that is detectable in and compatible with images formed by at least two imaging modalities, wherein one of the imaging modalities comprises a magnetic field imaging modality;
forming at least one image of the anatomical site, in which the marker is detectable and compatible, to obtain information about the anatomical site; and
treating the anatomical site using the information obtained from the at least one image of the anatomical site.

2. The method of claim 1 wherein the magnetic field imaging modality comprises a magnetic resonance imaging modality.

3. The method of claim 1 wherein the marker does not cause substantial spectral distortion under MRS.

4. The method of claim 1 wherein one of the imaging modalities comprises a non-magnetic field imaging modality.

5. The method of claim 4 wherein the non-magnetic field imaging modality comprises a radiation imaging modality or an ultrasound imaging modality.

6. The method of claim 5 wherein the radiation imaging modality comprises an X-ray imaging modality.

7. The method of claim 6 wherein the X-ray imaging modality comprises fluoroscopy or mammography.

8. The method of claim 1 wherein one of the imaging modalities comprises a magnetic resonance imaging modality and one comprises an X-ray imaging modality or an ultrasound imaging modality.

9. The method of claim 1 wherein the marker is detectable in and compatible with images formed by at least 3 imaging modalities.

10. The method of claim 9 wherein one of the imaging modalities comprises an ultrasound imaging modality and one comprises a radiation imaging modality.

11. The method of claim 10 wherein the radiation imaging modality comprises an X-ray imaging modality.

12. The method of claim 11 wherein the X-ray imaging modality comprises fluoroscopy or mammography.

13. The method of claim 9 wherein one of the imaging modalities comprises a magnetic resonance imaging modality, one comprises an ultrasound imaging modality and one comprises a radiation imaging modality.

14. The method of claim 1 wherein treating the anatomical site comprises monitoring the anatomical site using information obtained from the at least one image.

15. The method of claim 1 wherein treating the anatomical site comprises mapping the anatomical site using information obtained from the at least one image.

16. The method of claim 1 wherein treating the anatomical site comprises performing radiation therapy, drug therapy or surgery at the anatomical site.

17. The method of claim 1 where treating the anatomical site comprises performing a tissue removal or biopsy procedure.

18. The method of claim 1 wherein treating the anatomical site comprises evaluating the anatomical site after performing a medical procedure on the anatomical site.

19. The method of claim 1 wherein the marker is implanted at the anatomical site before, after or during a tissue removal or biopsy procedure.

20. The method of claim 1 wherein the implanting step comprises guiding the marker to the anatomical site by forming at least one image using an ultrasound, radiation or magnetic field imaging modality.

21. The method of claim 1 wherein the implanting step comprises implanting a plurality of markers into a body comprising the anatomical site, wherein at least one of the markers is implanted at the anatomical site.

22. The method of claim 1 wherein at least one image is formed by a magnetic field imaging modality, a radiation imaging modality or an ultrasound imaging modality.

23. The method of claim 1 further comprising forming at least a second image of the anatomical site, in which the marker is detectable in and compatible with, to obtain information about the anatomical site.

24. The method of claim 23 wherein at least one of the images is formed by magnetic resonance imaging.

25. The method of claim 23 wherein the second image is formed before, during or after treating the anatomical site.

26. The method of claim 1 wherein the information obtained from the at least one image comprises diagnostic information, positional information, or condition information about the anatomical site.

27. The method of claim 1 wherein the marker further comprises an additional material visible in an additional imaging modality.

28. The method of claim 27 wherein the additional imaging modality comprises an electronic portal imaging modality or a portal film imaging modality.

29. The method of claim 1 wherein the marker further comprises a biologically active agent.

30. The method of claim 1 further comprising injecting a carrier solution at the anatomical site before, after or during the implantation of the marker.

31. The method of claim 30 wherein the carrier solution comprises a glucan, collagen, saline, dextran, glycerol, polyethylene glycol, corn oil, safflower, polysaccharide, biocompatible polymer, methyl cellulose, agarose, hemostatic agent, protein or combinations thereof.

32. The method of claim 30 wherein the carrier solution comprises a β-glucan.

33. A method for mapping a portion of a body by multi-modality fusion comprising:
implanting at least one permanent tissue marker in the body, the marker comprising a solid material that is detectable in and compatible with images formed by at least two imaging modalities, wherein one of the imaging modalities comprises a magnetic field imaging modality;
forming a first image, in which the marker is detectable and compatible, using a first imaging modality;
forming a second image, in which the marker is detectable and compatible, using a second imaging modality, wherein one of the first and second imaging modalities is a magnetic field imaging modality; and
synthesizing the first and second images to obtain positional information for a portion of the body.

34. The method of claim 33 wherein the synthesizing step comprises synthesizing the first and second images using a computer system.

35. A method of positioning a body for radiation therapy comprising:
selecting an anatomical site upon which radiation therapy is to be performed;
implanting at least one permanent marker at the anatomical site, the marker comprising a solid material that is detectable in and compatible with images formed by at least two imaging modalities, wherein one of the imaging modalities comprises a magnetic field imaging modality;
forming at least one image of the anatomical site, in which the marker is detectable and compatible, to obtain information about the anatomical site; and
positioning the body for radiation therapy based on information provided by the at least one image.

36. The method of claim 35 further comprising:
forming at least two images of the anatomical site, in which the marker is detectable and compatible, to obtain information about the anatomical site; and
comparing information provided by the at least two images prior to performing radiation therapy.

37. The method of claim 36 wherein the comparing step comprises detecting positional differences between the at least two images.

38. The method of claim 36 comprising affecting the position of the patient based on the positional differences between the images.

39. The method of claim 35 further comprising pre-positioning the body for radiation therapy prior to forming the at least one image.

40. The method of claim 35 wherein the forming and positioning steps are performed at a plurality of radiation therapy sessions.

41. The method of claim 35 further comprising performing radiation therapy on the anatomical site.

42. A method of identifying a lesion site of a breast for treatment comprising:
implanting a marker at the lesion site comprising a solid material that is detectable in and compatible with images formed by at least two imaging modalities, wherein one of the imaging modalities comprises a magnetic field imaging modality;
forming at least one image of the lesion site, in which the marker is detectable and compatible, to obtain information about the lesion site; and
treating the lesion site using information obtained from the image.

43. The method of claim 42 wherein treating the lesion comprises monitoring the lesion.

44. The method of claim 42 wherein treating the lesion comprises removing the lesion from the breast.

45. The method of claim 42 wherein at least one image of the lesion site is formed by an MR mammography imaging modality.

46. A method for performing computer assisted diagnosis to provide diagnostic information about a patient comprising:
implanting at least one permanent marker at an anatomical site in the patient, the marker comprising at least one solid material that is not categorized as abnormal tissue during computer assisted diagnosis; and
performing computer assisted diagnosis to obtain diagnostic information about the anatomical site.

47. The method of claim 46 further comprising treating the anatomical site based on the diagnostic information.

48. The method of claim 47 wherein treating the anatomical site comprises performing radiation therapy.

49. A permanently implantable biocompatible marker comprising at least one solid material that is detectable in and compatible with images formed by at least two imaging modalities, wherein one of the at least two imaging modalities is a magnetic field imaging modality, and wherein the marker is shaped to be distinguishable from anatomical features in images formed by the imaging modalities.

50. The marker of claim 49 wherein the magnetic field measuring imaging modality comprises a magnetic resonance imaging modality.

51. The marker of claim 49 wherein the solid material is compatible with images formed by a radiation imaging modality or an ultrasound imaging modality.

52. The marker of claim 51 wherein the radiation imaging modality comprises X-ray.

53. The marker of claim 52 wherein the X-ray imaging modality comprises fluoroscopy or mammography.

54. The marker of claim 49 wherein the solid material is detectable in and compatible with images formed by at least 3 imaging modalities.

55. The marker of claim 54 wherein one of the imaging modalities comprises a radiation imaging modality and one comprises ultrasound.

56. The marker of claim 55 wherein the radiation imaging modality comprises an X-ray imaging modality.

57. The marker of claim 49 wherein the solid material comprises a ceramic material or graphite.

58. The marker of claim 57 wherein the ceramic material comprises zirconium oxide.

59. The marker of claim 57 wherein the ceramic material comprises aluminum oxide, hydroxyapatite, silicon dioxide or combinations thereof.

60. The marker of claim 49 wherein the solid material is coated with a biocompatible coating.

61. The marker of claim 60 wherein the biocompatible coating comprises a carbon coating or a carbon resin coating.

62. The marker of claim 61 wherein the carbon coating comprises pyrolytic carbon, vitreous carbon or graphite.

63. The marker of claim 49 comprising a zirconium oxide substrate and a carbon coating.

64. The marker of claim 49 comprising a major dimension between about 80 and about 10,000 microns.

65. The marker of claim 49 comprising a major dimension between about 800 and about 3,500 microns.

66. The marker of claim 49 comprising a major dimension between about 1,000 and about 3,000 microns.

67. The marker of claim 49 wherein the marker is shaped as a dog bone, barbell, ring, helix, tube, circle, oval or sphere.

68. The marker of claim 49 wherein the marker comprises a hollow portion.

69. The marker of claim 68 wherein the hollow portion is filled with a liquid.

70. The marker of claim 49 further comprising an additional material detectable in and compatible with at least an additional imaging modality.

71. The marker of claim 70 wherein the additional material comprises a radiopaque material.

72. The marker of claim 70 wherein the additional material comprises gold, titanium, platinum, palladium, gadolinium, or tantalum.

73. The marker of claim 70 wherein the additional material is applied as a coating.

74. The marker of claim 49 wherein the marker further comprises a biologically active agent disposed on a surface of the marker.

75. The marker of claim 74 wherein the biologically active agent is a biologically active gel.

76. The marker of claim 74 wherein the biologically active agent is an anti-inflammatory, anti-microbial, a hemostatic agent, a biocompatible adhesive agent, or a cell-derived agent.

77. A kit for marking an anatomical site comprising:
at least one marker for permanent implantation into the anatomical site comprising a solid material that is detectable in and compatible with images formed by at least two imaging modalities, wherein one of the imaging modalities is a magnetic field imaging modality, and wherein the marker is shaped to be distinguishable from features of the anatomical site; and
a carrier solution for delivery to the anatomical site.

78. The kit of claim 77 wherein the carrier solution comprises β-glucan.
